(19) United States
(12) Patent Application Publication
(10) Pub. No.: US 2016/0113963 A1
(43) Pub. Date: Apr. 28, 2016

(54) Method, Composition and System for Treatment of Irritations of Skin

(71) Applicant: Marirose Charlene Lynch, Eastham, MA (US)

(72) Inventor: Marirose Charlene Lynch, Eastham, MA (US)

(21) Appl. No.: 14/856,206

(22) Filed: Sep. 16, 2015

Related U.S. Application Data
(60) Provisional application No. 62/051,635, filed on Sep. 17, 2014.

(51) Int. Cl. A61K 33/30; A61K 47/44; A61K 9/00; A61K 33/26; A61K 33/22; A61K 31/12; A61K 47/06

(52) U.S. Cl. CPC A61K 33/30; A61K 31/12; A61K 47/44; A61K 47/06; A61K 33/26; A61K 33/22; A61K 9/0014

(57) ABSTRACT

A composition and system for the treatment of irritations of the skin includes therapeutically effective amounts of tincture benzoin and zinc oxide; a therapeutically effective amount of Castor oil, used as an epidermal permeation enhancer; and a therapeutically effective amount of petroleum jelly used as a moisturizing agent.

### REFERENCE TO RELATED APPLICATION

This application claims the benefit under 35 USC 119(e) of provisional patent Application Ser. No. 62/051,635, filed Sep. 17, 2014, which is incorporated by reference in its entirety.

### BACKGROUND OF THE INVENTION

1. Field of the Invention

This invention relates to an improved and useful topical system for the alleviation of a variety of skin conditions inclusive of itching, dermatitis, cracking, burning and peeling of the skin.

2. Prior Art

The identification and, therefore, the formulation of a suitable composition which could form the basis of an over-the-counter (OTC) treatment of a variety of irritations of the skin and therefore not requiring the prescription of a physician has been sought for many years. The present invention relates to a composition and method which, when produced and used in the manner and fashion set forth herein yields a synergistic effect between its ingredients and is useful upon a variety of dermatologic conditions including, but not limited to the above set forth conditions.

An important aspect of the present invention relates to the restoration of the natural turgidity or flexibility of the skin which, generally, is considered related to the lipid layer of the dermis. Turgidity is considered essential for the health of the dermis.

In terms of the prior art, the present method and composition relates to the use of zinc oxide in both its semi-solid form and in a liquid emulsion known as Calamine lotion when mixed with about 0.5% of ferric oxide or zinc carbonate.

The invention also, as is more fully set forth below, employs a quantity of tincture of benzoin which, in the prior art, has found many uses including as a styptic (to reduce bleeding) and as an antiseptic. A resin of benzoin is typically mixed with alcohol to form the product known as tincture of benzoin. Its use has been employed for the treatment of hemorrhoids as is taught in U.S. Pat. No. 4,169,143 (1979) to Haimowitz, and for the removal of blemishes of the face as is taught by U.S. Pat. No. 5,431,911 (1995) to Reynolds. Various moisturizing agents have been mixed with tincture of benzoin to address alleviation of many conditions including dermatitis and dry, cracked, flaky, and peeling condition of the skin, particularly of the foot, as is taught in U.S. Application No. 2001/0005509 (2001) to Harbeck.

The instant invention also employs the skin-penetrating property found to exist in Castor oil, given that many conditions of the skin cannot be treated unless the composition is able to penetrate to layers of the skin beneath the epidermis. Castor oil, occasionally mixed with other oils, such as coconut oil and soy bean, have been employed to assist in the administration of transdermally delivered prescription drugs as is taught by U.S. Pat. No. 5,229,130 (1993) to Sharma. Castor oil per se is a vegetable oil obtained by the pressing of seeds of the Castor oil plant (Ricinus Communis). Castor oil is colorless, pale yellow liquid having a particular high boiling point for a liquid, namely, 595 degrees Fahrenheit. As such, it is suitable for uses in methods and formulations requiring a heating thereof in the neighborhood of 200 degrees Fahrenheit as is taught herein.

In light of the above, the invention may be viewed as an improvement of historic Calamine which itself is over 99% zinc oxide but in the form of an emulsion within a pharmaceutically acceptable carrier. Calamine did not receive FDA approval as an OTC topical skin product to relieve itching caused by poisonous plants including poison ivy, poison oak and poison somac until 2008. Before then, over 400 articles published questioning the value of Calamine as safe or effective in the treating of bug bites, stings or rashes from poisonous plants. In this light, the present invention may be viewed as a significant improvement of Calamine which has never been accepted in the medical community for any purpose other than to relieve itching caused by poisonous plants.

### SUMMARY OF THE INVENTION

A method and system for forming a composition for the treatment of irritations of the skin includes the steps of mixing about 40 to about 80 milliliters of Calamine, with about 20 to about 40 milliliters of tincture of benzoin, and then with about 40 to about 80 milliliters of Castor oil. Into the above mixture is stirred between about 100 and about 130 grams of petroleum jelly and about 40 to about 80 grams of -zinc oxide ointment of a concentration of about 15 to about 25 percent. The above ingredients are then heated and stirred until they have melted together in a single mass, such heating occurring at temperatures preferably under 200 degrees Fahrenheit. The resulting composition may then be emulsified into a liquid form or may be mixed with a pharmaceutically acceptable carrier to produce a semi-solid ointment.

It is accordingly an object of the instant invention to provide a skin treatment composition effective in the treatment of irritations associated with various dermatologic conditions of the skin.

It is another object to provide a skin treatment formulation which soothes and moisturizes the skin to remediate the effects of itching and other forms of irritation.

It is a further object of the invention to provide an enhancement of traditional Calamine and zinc oxide to effect the removal of skin blemishes and to restore vital nutrients, softness and flexibility to the skin.

It is a yet further object to provide a method and treatment of the skin that may be readily formulated without requirement of costly ingredients, complicated preparation procedures, or FDA approval for OTC use.

It is another object of the invention to provide a composition, for treatment of irritations of the skin, having increased capacity of sub-dermal penetration thereof.

It is a further object to provide a skin treatment composition which avoids the use of complex synthetic adjuvants for use in its formulation.

It is a yet further object to provide a method and composition for the treatment of distressed skin conditions which minimizes allergic skin reactions which occur with the use of many synthetic compounds and adjuvants.

It is a yet further object to provide a composition and method for the preparation thereof for the treatment of itching and irritating skin conditions while providing anti-inflammatory qualities, moisturizing and softening of the skin, elimination or reduction of dryness, cracking, chapping, redness and the like to thereby restore the skin to a natural healthy condition.

It is another object to provide an alternative skin therapy applicable in the fields of dermatology and cosmetology.

The above and yet other objects and advantages of the present invention will become apparent from the hereinafter set forth Detailed Description of the Invention and Claims appended herewith.

### DETAILED DESCRIPTION OF THE INVENTION

The present composition for the treatment of irritations of the skin and the general soothing thereof includes the following:

1. Therapeutically effective amounts of tincture of benzoin and of zinc oxide.

2. A therapeutically effective amount of Castor oil employed as an enhancer of permeation of the epidermis.

3. A therapeutically effective amount of petroleum jelly used as a moisturizing agent to act synergistically with said Castor oil for the moisturizing and penetration of the epidermis.

In the present invention, zinc oxide takes two essential forms, namely, zinc oxide ointment which is an organic compound derived from metallic zinc which was known in ancient Indian and Greek medicine as a preferred treatment for a variety of skin conditions, and today is a common component in anti-dandruff shampoos.

Zinc oxide is also used today as the primary component (over 99%) of Calamine lotion in which the zinc oxide is emulsified with a small quantity of ferric oxide.

Zinc oxide first appeared in Europe in 1743 as a byproduct of smelting of zinc and was produced as a pigment used in oil paintings. However, its medicinal advantages were soon recognized such that by 1850 ointment of zinc oxide was widely manufactured throughout Europe. As noted, zinc oxide, when mixed with an emulsified together with a small quantity of Ferric oxide produces the product popularly known as Calamine. Zinc ointment also appears in creams and lotions and constituted the first FDA-approved product against sun burns and damage to the skin caused by ultraviolet light. More recently, nano-particles of zinc oxide have been found to enhance the anti-bacterial activity of Ciprofloxacin, a contemporary antibiotic used to treat various infections. When emulsified into the form of the Calamine product, Calamine was approved as a topical OTC skin treatment for the relief of itching caused by various plants.

Notwithstanding the limited acceptance of Calamine, the present invention combines the basic ingredients of Calamine and its zinc oxide, both in emulsified and non-emulsified forms, in combination with tincture of benzoin, Castor oil, and petroleum jelly as a means of treating many forms of skin irritation and clearing of blemishes of the face. As above noted, tincture of benzoin, which is a solution of a benzene resin in alcohol, has long been recognized for its antiseptic effects and as a skin cleanser. It is also recommended for application to areas of irritation of the skin to protect such areas from further irritation until they are able to heal through the effects of other agents. Benzoin itself is a complex organic molecule, discovered in 1832 during research concerning the properties of almond nuts. Benzoin is also used in aromatherapy and provides a vanilla-like odor. Its therapeutic properties as an anti-bacterial, anti-fungal, sealant of infectious wounds, promotion of circulation and as an anti-oxidant have been documented. Cream of benzoin has been used to protect chapped hands and improve skin elasticity. As such, tincture of benzoin has been recognized for its various medicinal properties while the benefit of Calamine lotion and its constituent zinc oxide lies mainly in its soothing effect, particularly as remedial to conditions of itching. To the knowledge of the inventor, tincture of benzoin has never been combined with zinc oxide in any form.

The effect of petroleum jelly in the present formulation is to provide suppleness to otherwise chapped damaged or irritated skin, while the function of Castor oil as set forth herein is to facilitate the sub-dermal penetration of the composition to all layers of the skin, given that many skin conditions operate more deeply than simply the epidermis or outer layer of the skin.

In an alternate embodiment of the invention, Boric Acid many be employed as a substitute for tincture of benzoin, given the many healing properties.

In summary, the inventive composition and method exhibits three basic components, namely:

1. The therapeutically active elements thereof, namely, tincture of benzoin and zinc oxide.

2. The epidermal penetration enhancer, namely, Castor oil.

3. The general moisturizing agent of petroleum jelly which, to some extent, is enhanced by zinc oxide, whether alone or in the form of Calamine lotion.

In the inventive composition, a quantity of tincture of benzoin in the range of about 20 to about 40 milliliters is used. In regard to Castor oil, a quantity of between about 40 and about 80 milliliters is employed.

In regard to petroleum jelly, a quantity by weight of between about 90 and about 140 grams is employed. In the use of a zinc oxide ointment, a concentration in the range of 15 to about 25 percent is used in a weight of semi-solid zinc oxide in a range of about 40 to about 80 grams. Finally, a volume of Calamine lotion in a range of about 40 to about 80 milliliters is employed.

In terms of the method of forming the composition described, the method is defined by the steps of mixing about 40 to about 80 milliliters of Calamine lotion, with about 20 to about 40 milliliters of tincture of benzoin, with about 40 to about 80 milliliters of Castor oil. Into the above mixture is stirred between about 100 to about 130 grams of petroleum jelly and about 40 to about 80 grams of a concentration of zinc oxide ointment of about 15 to about 25 percent. The above ingredients are then heated and stirred until they have melted together into a single viscous mass, such heating occurring at temperatures preferably under 200 degrees Fahrenheit. The resulting composition may then be emulsified into a liquid form or may be mixed with a pharmaceutically acceptable carrier to produce a semi-solid ointment.

In an alternate embodiment, between 5 and 15 ml of Boric Acid may be added to the composition.

While there has been shown and described above the preferred embodiment of the instant invention it is to be appreciated that the invention may be embodied otherwise than is herein specifically shown and described and that, within said embodiment, certain changes may be made in the form and arrangement of the parts without departing from the underlying ideas or principles of this invention as set forth in the Claims appended herewith.

I claim:

1. A composition and system for the treatment of irritations other conditions of the skin, comprising:
(a) therapeutically effective amounts of tincture of benzoin and zinc oxide;
(b) a therapeutically effective amount of Castor oil, as an epidermal permeation enhancer; and
(c) a therapeutically effective amount of petroleum jelly, as a moisturizing agent.

2. The composition as recited in claim 1, in which said zinc oxide comprises:
a part of Calamine lotion into which the zinc oxide is emulsified into a liquid form.

3. The composition as recited in claim 2, in which said tincture of benzoin defines about 20 to about 40 milliliters within the composition.

4. The composition as recited in claim 3, in which Castor oil defines about 40 to about 80 milliliters of the composition.

5. The composition as recited in claim 4, in which said petroleum jelly comprises, by weight, about 90 to about 140 grams within the composition.

6. The composition as recited in claim 2, in which said zinc oxide, in solid or semi-solid form, defines:
a concentration of about 15 to about 25 percent within a weight of about 40 to about 80 grams of zinc oxide within the composition; and
said Calamine lotion defines a volume of about 40 to about 80 milliliters.

7. The composition as recited in claim 6, in which said tincture of benzoin defines about 20 to about 40 milliliters within the composition.

8. The composition as recited in claim 6, in which Castor oil defines about 40 to about 80 milliliters of the composition.

9. The composition as recited in claim 8, in which said petroleum jelly comprises, by weight, about 90 to about 140 grams within the composition.

10. The composition as recited in claim 6, in which said Calamine lotion includes a concentration of about 0.5% of ferric oxide.

11. The composition as recited in claim 6, further including the addition of between 5 and 15 milliliters of Boric Acid.

12. A method and system of forming a composition for the treatment of irritations and blemishes of the skin, the method comprising the steps of:
(a) mixing about 40 to about 80 milliliters of Calamine lotion with about 20 to about 40 milliliters of tincture of benzoin, and with about 40 to about 80 milliliters of Castor oil;
(b) stirring the above mixture about 100 to about 130 grams of petroleum jelly and about 40 to about 80 grams of zinc oxide ointment having a concentration of about 15 to about 25 percent; and
(c) heating and stirring the above components until they have melted together in a viscous mass, said heating occurring at temperatures at less than 100 degrees C.

13. The method as recited in claim 12, further including the step of:
(d) emulsifying the composition resultant of Step (c) above; and
(e) using a suitable preservative and storing the resultant composition in the form of a semi-solid ointment.

14. The method as recited in claim 12, further comprising the step of:
emulsifying the composition resultant of Step (c) into the form of a lotion.
